(19) United States
(12) Patent Application Publication
(10) Pub. No.: US 2018/0228407 A1
(43) Pub. Date: Aug. 16, 2018

(54) SYSTEM FOR HAND REHABILITATION

(71) Applicant: THE JOHNS HOPKINS UNIVERSITY, Baltimore, MD (US)

(72) Inventors: Kevin C. Olds, Baltimore, MD (US); Omar Ahmad, Baltimore, MD (US); Promit Roy, Baltimore, MD (US); John Krakauer, Baltimore, MD (US)

(21) Appl. No.: 15/897,756

(22) Filed: Feb. 15, 2018

Related U.S. Application Data
(60) Provisional application No. 62/459,892, filed on Feb. 16, 2017.

(51) Int. Cl. A61B 5/11; A61B 5/00; A63F 13/24

(52) U.S. Cl. CPC A61B 5/1125; A61B 5/486; A61B 2090/064; A63F 13/24; A61B 5/6826

(57) ABSTRACT

Provided is a system for hand rehabilitation comprising an enclosure comprising a top surface and a bottom surface opposite the top surface; a wrist support attached to the top surface and configured to support and rigidly hold a wrist in position with respect to the top surface; a finger retention support arranged on the top surface, sized to accommodate at least a fingertip, and arranged to allow the finger to exert forces on the finger retention support in any direction; a sensor housed within the enclosure and in communication with the finger retention support, the sensor configured to detect and convert forces exerted by the finger on the finger retention support in multiple degrees of freedom into an electrical signal; and a processor in electrical communication with the sensor and configured to receive the electrical signal and provide feedback to a user.

### CROSS REFERENCE TO RELATED APPLICATIONS

The present application claims benefit of U.S. Provisional Application No. 62/459,892 filed on Feb. 16, 2017, which is hereby incorporated by reference in its entirety.

### FIELD

The present disclosure relates to a system for hand rehabilitation.

### BACKGROUND

In the modern world, interacting with computers has become a critical skill, however conventional computer interfaces like mice, keyboards, and touch screens are not accessible to everyone, particularly users with disabilities affecting their hands. One of the most common types of disabilities affecting computer use is motor-neurological injuries such as stroke, traumatic brain injury, spinal cord injury, and peripheral nerve injury. These disabilities can make it difficult for users to control their hands and fingers, making them unable to exert sufficient force for a computer interface device, e.g., not enough finger strength to move a joystick, move over a sufficient range of motion for a computer interface device, e.g., not enough finger range of motion to reach everywhere on a touch screen, stabilize the arm/hand while doing finger movements, e.g., the arm and wrist are unstable, making it difficult to target points on a touch screen accurately with a finger, and inability to feel a sense of touch or position of the finger/hand, e.g., user is not able to feel where the keyboard keys are through touch alone, or whether their finger is currently pressing or not.

These problems are not limited to casual or occupational computer use. They also make it difficult for users to use computer-based technology to rehabilitate their own injuries. Several such computer-based technological solutions exist for hand/finger rehabilitation. FIG. 1 shows a Tyromotion Amadeo device. The Amadeo is a robotic rehabilitation system that uses linear actuators to both move the user's fingers and also detect user-generated finger motion. Components of the Amadeo include a brace attached to the device for stabilizing the arm and wrist, magnets held on the finger tips by adhesive strips, which magnetically couple the user's fingers to the linear actuators, force sensors to detect user input, a computer running rehabilitation software including games for rehabilitation, and a display to give feedback to the user.

The Amadeo also has several drawbacks including the setup time being long due to the difficulty of setting up the adhesive strips and magnets, making it difficult for users to use the device unsupervised, the device is large and not easily portable, the force sensors can be used to initiate a motion or assess a patient, but cannot be used to continuously control the device, the friction of the linear actuators makes moving the device difficult for weak users, and the fingers can only move back and forth in one direction.

FIG. 2 shows a Rapael Smart Glove by Neofect that is another conventional device used for hand rehabilitation. The Rapael uses a flexible, wearable frame combined with bend sensors to detect finger motion. The Rapael has several drawbacks including weak patients may have difficulty overcoming the stiffness of the device to move their fingers and the fingers may only detect motion back and forth in one direction, which excludes many natural finger movements.

FIG. 3 shows another type of device used for finger rehabilitation is the robotic exoskeleton, as described by S. Ito, H. Kawasaki, Y. Ishigure, Y. Nishimoto, T. Aoki, T. Mouri, H. Sakaeda and M. Abe, “Development of a Hand Motion Assist Robot for Rehabilitation Therapy by Patient Self-Motion Control,” in Proceedings of the 2007 IEEE 10th International Conference on Rehabilitation Robotics, Noordwijk, Netherlands, 2007 (“S. Ito”). This device uses multiple 3-axis force sensors to detect forces from along the back of the user's fingers. This force data is then used to drive the robotic exoskeleton to comply with the user's motion. This approach has several advantages compared to the above-described prior approaches, including capturing a broad range of finger movements and the ability to comply with weak user motions. However, it also has many disadvantages, including the need for multiple force sensors per finger, resulting in high cost, requiring the user to put on a glove, and many patients with impaired hand function have great difficulty putting on a glove, particularly if they have difficulty keeping their fingers straight, which is a common problem. Also, the device is heavy and is not easily portable and the device is complex with many potential risks such as pinch points, making unsupervised use nearly impossible.

FIG. 4 shows another type of device, which is a finger keyboard described in J. Xu, N. Ejaz, B. Hertler, M. Branscheildt, M. Widmer, A. Faria, M. Harran, J. Cortes, N. Kim, P. Celnik, T. Kitago, A. Luft, J. Krakauer and J. Diedrichsen, “Recovery of hand function after stroke: separable systems for finger strength and control,” bioRxiv, 2016 (“J.Xu'”). This device was used to measure finger forces for scientific studies. This device includes a basic wrist support, force sensing keys that can detect pressing force in one direction for each finger, and elastic hook-and-loop straps on each key which prevent the fingers from sliding off. It is also relatively light and portable. One drawback of this device is that it can only detect finger force in one direction, preventing measurement of a wide range of possible finger movements.

Another prior art device is described in U.S. Pat. No. 6,673,026 to D R. Pozos and J. Agraz titled “Force measuring device and method,” which discloses a device similar to the one in J.Xu, though this device has no means of keeping the finger on the force sensor and does not discuss detecting forces in multiple axes for each finger.

A different approach is described in U.S. Pat. No. 6,622,575 to K. Nagata titled “Fingertip-mounted six-axis force sensor,” which describes using six-degree-of-freedom force sensors integrated into a fingertip-mounted thimble-like device. However, this device's main use is detecting finger forces during natural grasping, and it is not well suited for rehabilitation due to its lack of arm/wrist support and free-floating sensors.

Thus, a heretofore unaddressed need exists in the industry to address the aforementioned deficiencies and inadequacies.

### SUMMARY

In accordance with examples of the present disclosure, a system for hand rehabilitation is provided. The system comprises an enclosure comprising a top surface and a bottom surface opposite the top surface; a finger retention support arranged on the top surface, sized to accommodate at least a fingertip, and arranged to allow a finger to exert directional movement in three degrees of freedom; and a first force sensor housed within the enclosure and configured to detect and convert the directional movement exerted by the finger to one or more electrical control signals.

In some examples, the system can further comprise a wrist support attached to the top surface and configured to support and rigidly hold a wrist in position with respect to the top surface and a processor in electrical communication with the first force sensor and configured to receive the one or more electrical control signals from the first force sensor and provide feedback to a user. In some examples, the system can further comprise a printed circuit board (PCB) housed within the enclosure, wherein the first force sensor is directly mounted on the PCB. In some examples, the system can further comprise a second force sensor, a third force sensor, and a fourth force sensor mounted directly on the PCB. In some examples, the first force sensor, the second force sensor, the third force sensor, and the fourth force sensor are mounted on a top surface of the PCB. In some examples, the system can further comprise a fifth force sensor, a sixth force sensor, a seventh force sensor, and an eighth force sensor mounted directly on a bottom surface of the PCB. In some examples, the first force sensor, the second force sensor, the third force sensor, and the fourth force sensor are strain gauges. In some examples, the system can further comprise a display configured to display the feedback. In some examples, the processor is configured to perform operations based on movements of the finger as measured by the first strain gauge. In some examples, the feedback comprises an interactive video or video game. In some examples, the finger retention support comprises an elastic silicon cup bonded onto the top surface, a passive retention support, a pneumatic powered suction cup, a rigid support with one or more elastic or spring loaded parts which exert pressure on the finger, a rigid support that can be mechanically adjusted in tightness around the finger such as a by a collet mechanism or a screw/band clamp mechanism, or a support with removable adhesive. In some examples, the finger retention support comprises one finger retention support for each finger. In some examples, the first force sensor is configured to detect bi-directional forces exerted by the finger on the finger retention support in multiple degrees of freedom.

According to examples of the present disclosure, a system for hand rehabilitation is provided. The system can comprise an enclosure comprising a top surface and a bottom surface opposite the top surface; an input device arranged on the top surface and arranged to allow a finger to exert directional movement in three degrees of freedom; and a first force sensor housed within the enclosure and configured to detect and convert the directional movement exerted by the finger to one or more electrical control signals.

In some examples, the system can further comprise a wrist support attached to the top surface and configured to support and rigidly hold a wrist in position with respect to the top surface and a processor in electrical communication with the first force sensor and configured to receive the one or more electrical control signals from the first force sensor and provide feedback to a user. In some examples, the system can further comprise a printed circuit board (PCB) housed within the enclosure, wherein the first force sensor is directly mounted on a top surface of the PCB. In some examples, the system can further comprise a second force sensor, a third force sensor, and a fourth force sensor mounted directly on the top surface of the PCB. In some examples, the system can further comprise a fifth force sensor, a sixth force sensor, a seventh force sensor, and an eighth force sensor mounted directly on a bottom surface of the PCB. In some examples, the first force sensor, the second force sensor, the third force sensor, and the fourth force sensor are strain gauges.

In some examples, in addition or alternately to the force sensor, the system can comprise an optical-based sensor, a thin film force sensor, or a load cell. The force sensor can comprise a resistive strain gauge integrated onto a printed circuit board substrate. The force sensor can comprises four pair of resistive stain gauges with each pair mounted on opposite sides of printed circuit board substrate in a half-bridge configuration. The feedback can comprise a vibration-based feedback, a temperature-based feedback, a pressure-based feedback, or electrical stimulation-based feedback. The system can further comprise additional force sensors arranged between the top surface and the wrist support. The finger retention support can comprise an elastic silicon cup bonded onto the top surface, a passive retention support, a pneumatic powered suction cup, a rigid support with one or more elastic or spring loaded parts which exert pressure on the finger, a rigid support that can be mechanically adjusted in tightness around the finger such as a by a collet mechanism or a screw/band clamp mechanism, or a support with removable adhesive. The finger retention support can comprise one finger retention support for each finger. The sensors can comprise position measurement sensors for detecting finger motion, such as encoders, potentiometers, joysticks, or optical sensors. The sensor can be configured to detect bi-directional forces exerted by the finger on the finger retention support in multiple degrees of freedom.

In some examples, the system can further comprise a first strain gauge, a second strain gauge, a third strain gauge, and a fourth strain gauge mounted directly on the PCB. In some examples, the first strain gauge, the second strain gauge, the third strain gauge, and the fourth strain gauge are mounted on a top surface of the PCB. In some examples, the system can further comprise a fifth strain gauge, a sixth strain gauge, a seventh strain gauge, and an eighth strain gauge mounted directly on the PCB. In some examples, the fifth strain gauge, the sixth strain gauge, the seventh strain gauge, and the eighth strain gauge mounted directly on a bottom surface of the PCB. In some examples, the system can further comprise additional force sensors arranged between the top surface and the wrist support. The additional force sensors can comprise position measurement sensors for detecting finger motion, such as encoders, potentiometers, joysticks, or optical sensors. The first strain gauge can be configured to detect bi-directional forces exerted by the finger on the finger retention support in multiple degrees of freedom.

### BRIEF DESCRIPTION OF THE DRAWINGS

The accompanying drawings, which are incorporated in and constitute a part of this specification, illustrate the present disclosure and together with the description, serve to explain the principles of the present disclosure.

FIG. 1 shows a Tyromotion Amadeo device.

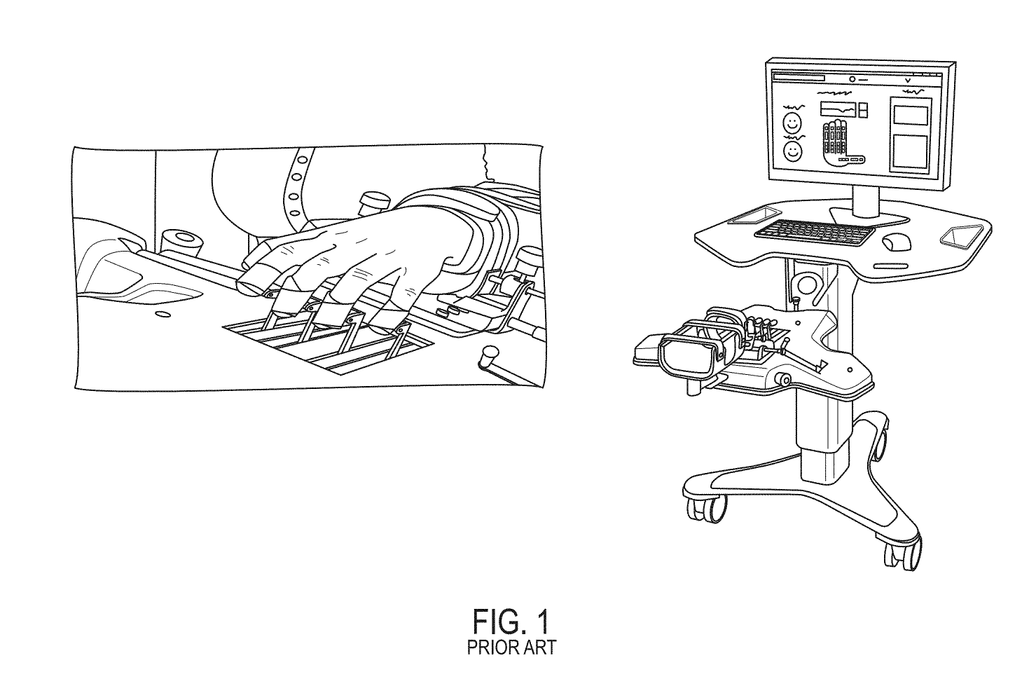

FIG. 2 shows a Rapael Smart Glove by Neofect.

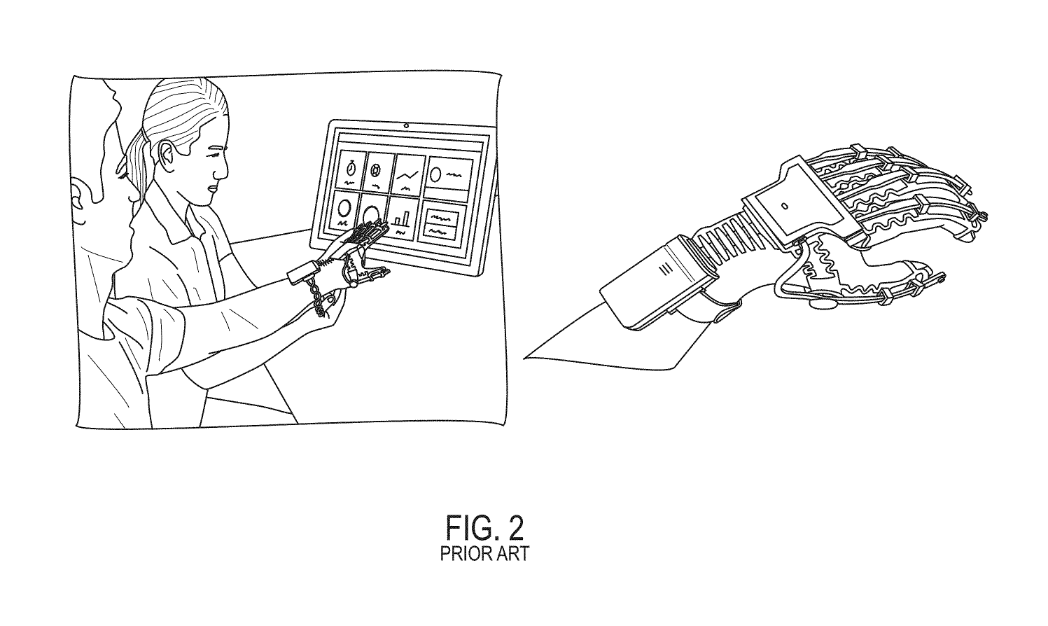

FIG. 3 shows another type of device used for finger rehabilitation is the robotic exoskeleton, as described by S. Ito.

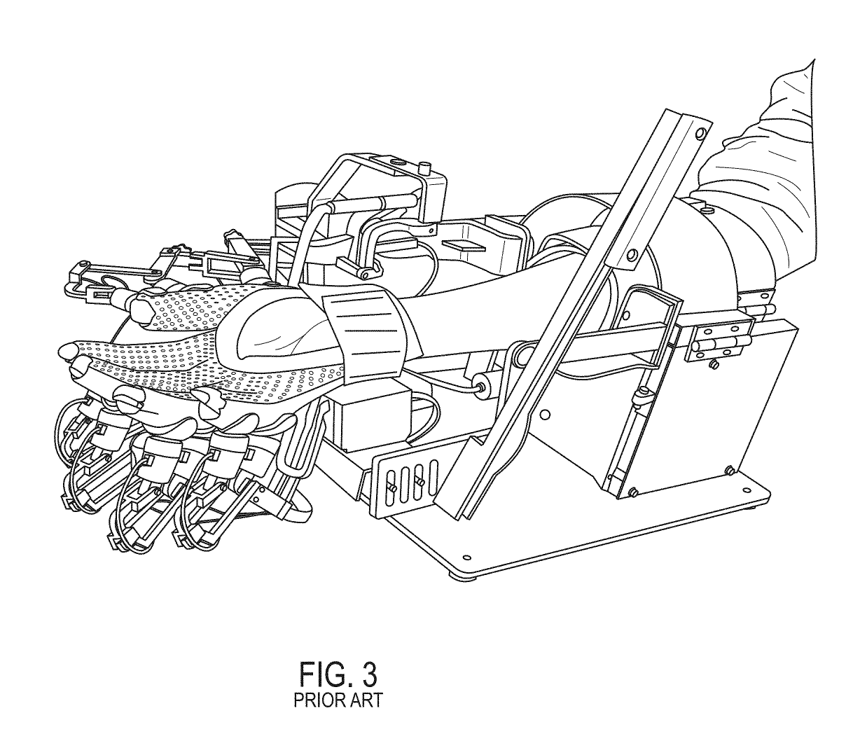

FIG. 4 shows another type of device, which is a finger keyboard described in J. Xu.

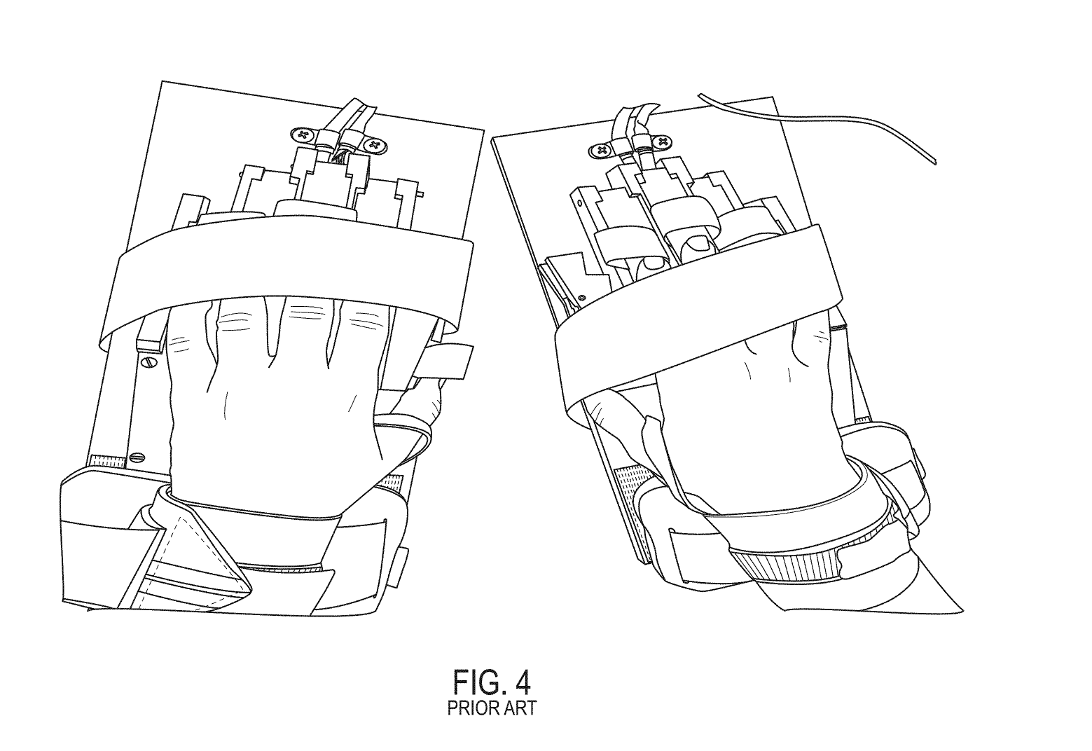

FIG. 5 shows a system for hand rehabilitation 500, according to examples of the present disclosure.

FIG. 6 shows the system 500 in use.

FIG. 7 shows the system 500 including a detachable cover 705 to improve ergonomics and cosmetics.

FIG. 8 shows an exploded view of the positioning mechanism 800, according to examples of the present disclosure.

FIG. 9 shows the interior of the chassis, including the electronics that process the signals from the force sensor, according to examples of the present disclosure

FIG. 10 shows the force sensor, according to examples of the present disclosure.

FIG. 11 illustrates a hardware configuration for computer device, which can be used to perform one or more of the processes disclosed herein, according to examples of the present disclosure.

### DESCRIPTION

Reference will now be made in detail to exemplary implementations of the present disclosure, examples of which are illustrated in the accompanying drawings. Wherever possible, the same reference numbers will be used throughout the drawings to refer to the same or like parts. In the following description, reference is made to the accompanying drawings that form a part thereof, and in which is shown by way of illustration specific exemplary implementations in which the present disclosure may be practiced. These implementations are described in sufficient detail to enable those skilled in the art to practice the present disclosure and it is to be understood that other implementations may be utilized and that changes may be made without departing from the scope of the present disclosure. The following description is, therefore, merely exemplary.

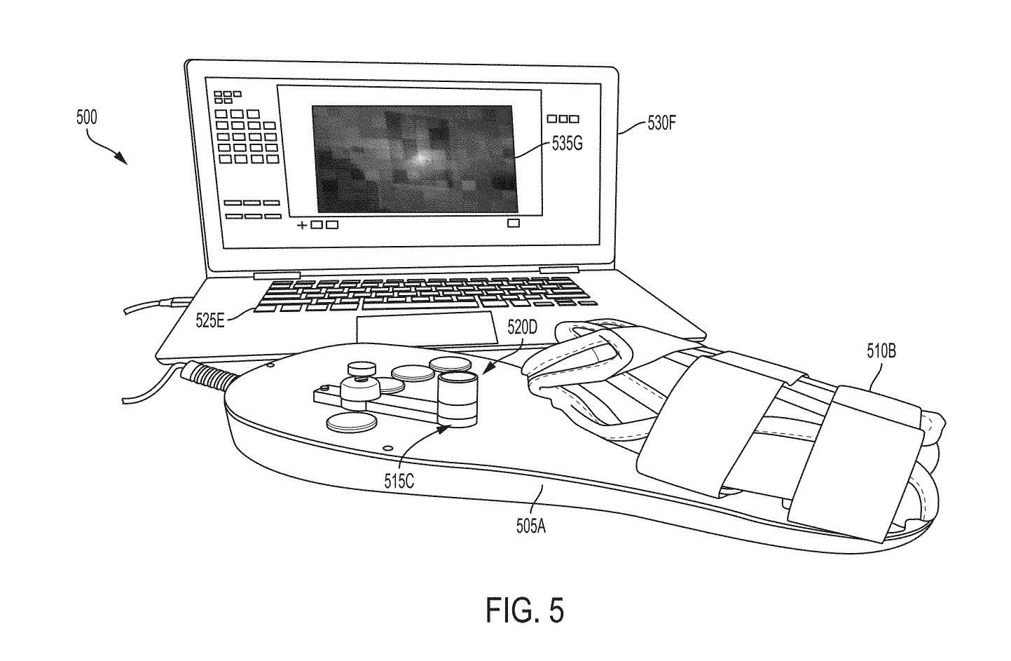

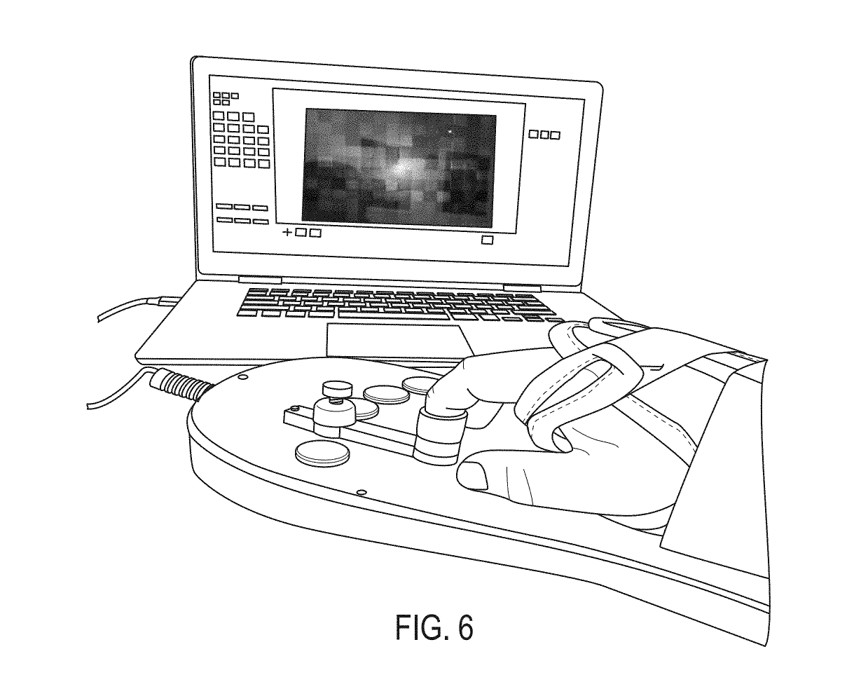

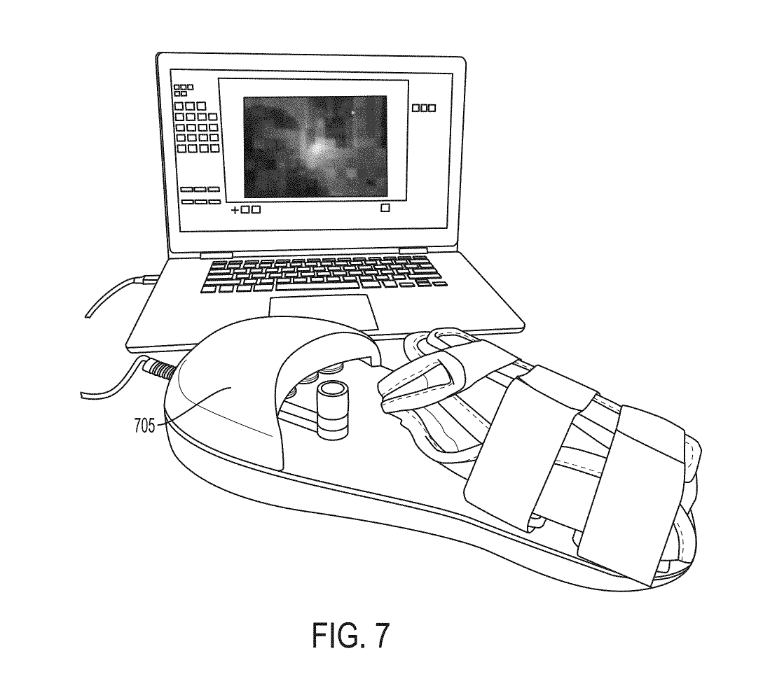

FIG. 5 shows a system for hand rehabilitation 500, according to examples of the present disclosure. FIG. 6 shows the system 500 in use. The system 500 comprises a base chassis 505, a wrist support 510 that supports and holds rigidly with respect to the base chassis, a multi degree-of-freedom (“dof”) bidirectional force sensors 515 that is rigidly attached to the base chassis 505 such that its position relative to the base chassis 505 can be adjusted to accommodate different hand sizes, finger retention adapters 520 that holds finger so force can be exerted in every direction, a computational processing unit 525, a display 530, and software 535 that encourages the user to rapidly explore diverse hand configurations. In some examples, the system 500 can include a detachable cover 705 to improve ergonomics and cosmetics, as shown in FIG. 7.

The base chassis 505 comprises a plastic and aluminum enclosure which both rigidly supports the wrist support 510 and the force sensors 515, and also contains the electronics which process sensor signals and communicate with the processing unit 525. The wrist support 510 supports the hand and holds it rigidly with respect to the base chassis. 505 The wrist support 510 aids in preventing unwanted arm/wrist movements from interfering with finger forces/movements by holding the wrist fixed. In one example, the force sensors 515 comprise resistive strain gauges integrated onto a printed circuit board substrate are used to detect finger forces. These force sensors 515 enable highly sensitive and reliable detection of bidirectional forces in three degrees of freedom. The finger retention adapters 520 comprise an elastic silicone cup bonded onto a rigid plastic base and are loose enough that a user can easily insert and remove their finger, but tight enough to support the fingertip through both friction and suction, enabling the finger to exert force in any direction (3 dof: into and out of the surface of the surface of the chassis, up, down, left, and right) without unintentionally coming out. In some examples, the processing unit 525 is a laptop computer that reads data from the force sensors 515 and generates outputs for the display 530. The screen of the laptop computer is used as the display 530, giving the user feedback related to their finger input. The rehabilitation gaming software 535 uses the sensor input to control the motion of a virtual character in the game.

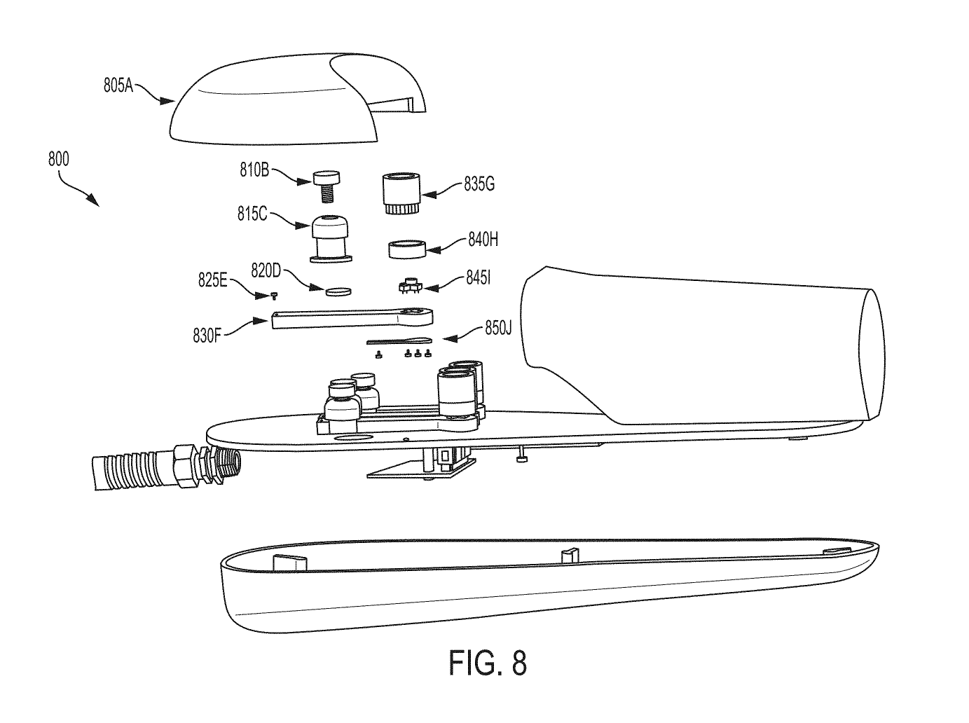

In order to accommodate users with different hand sizes/proportions, the force sensors 515 are mounted on adjustable positioning mechanisms which enable the force sensor 515 to be positioned in two degrees of freedom. FIG. 8 shows an exploded view of the positioning mechanism 800, according to examples of the present disclosure. The positioning mechanism 800 comprises a removable cover 805 for cosmetics/ergonomics, a thumb screw 810 for securing positioning mechanism in place, a turret 815 that contains force sensor rod while allowing its position to be adjusted in two degrees of freedom, a pressure disc 820 that prevents marring of the force sensor rod by the thumb screw, a retention screw 825 that keeps force sensor rod captive in the turret 815, a force sensor rod 830 that acts as a rigid frame for the force sensor, a finger retention adapter 835, a rigid base 840 for the finger retention adapter 835 that is threaded to allow easy mounting onto force sensor frame and allows for the finger retention adapter 835 to be quickly and easily changed, allowing different sizes to be used for different sized fingers, a force sensor frame 845 that is bonded onto the force sensor circuit board, and a force sensor circuit board 850 that contains the sensing elements. The top of the force sensor frame 845 can be a threaded shaft to receive the base of the finger retention adapter.

The position of the force sensor can be change by loosening the thumb screw 810 and moving the force sensor to the desired position, then tightening the thumb screw 810. The turret 815 is free to rotate through a hole in the chassis, and the force sensor rod 830 can slide through the hole in the turret 815, allowing two degrees of freedom of positioning. One aspect of the design of FIG. 8 is that it only requires one hand to set up the device, which can be important for users with impaired function in their other hand.

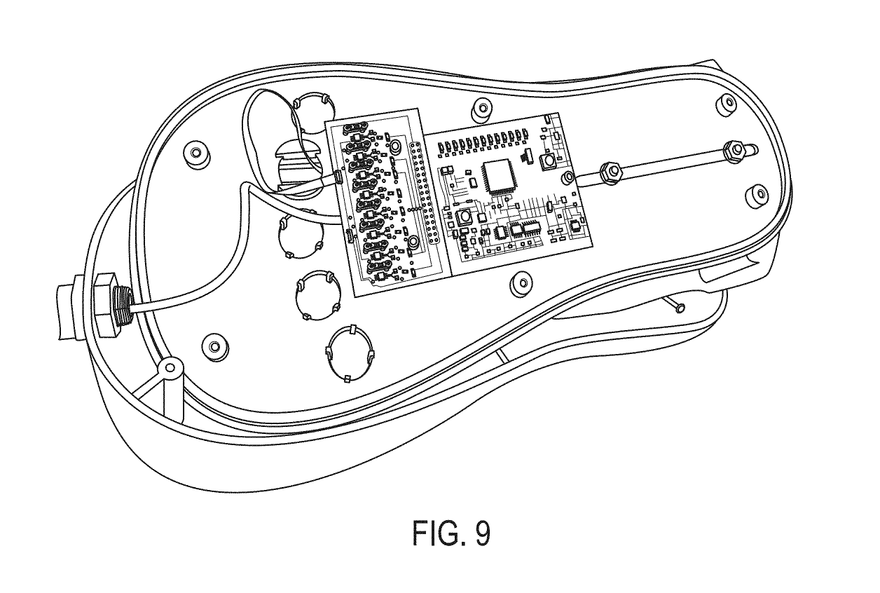

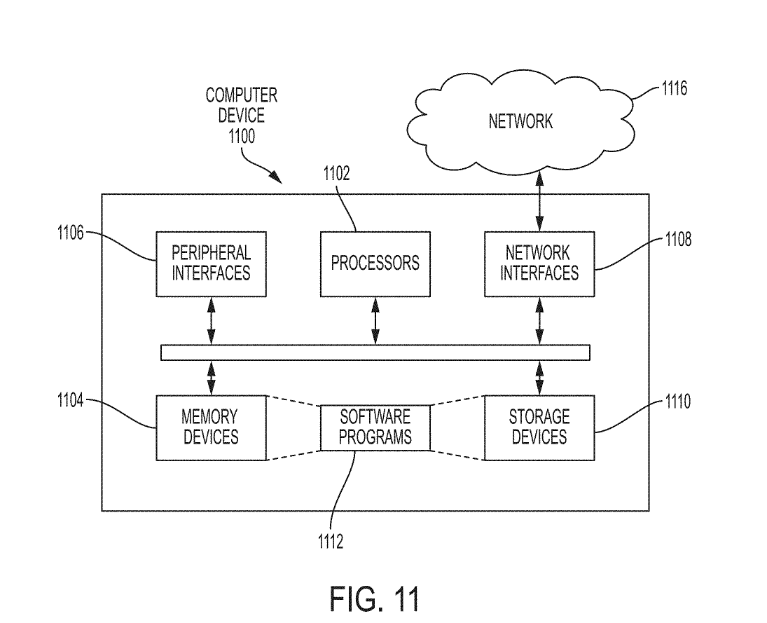

FIG. 9 shows the interior of the chassis, including the electronics that process the signals from the force sensor, according to examples of the present disclosure. The signals are passed from the force sensor to the electronics by a flex cable that passes through the hollow channel in the bottom of the force sensor rod. In this embodiment, the electronics communicate with the laptop PC (such as shown in FIG. 11) via a USB cable.

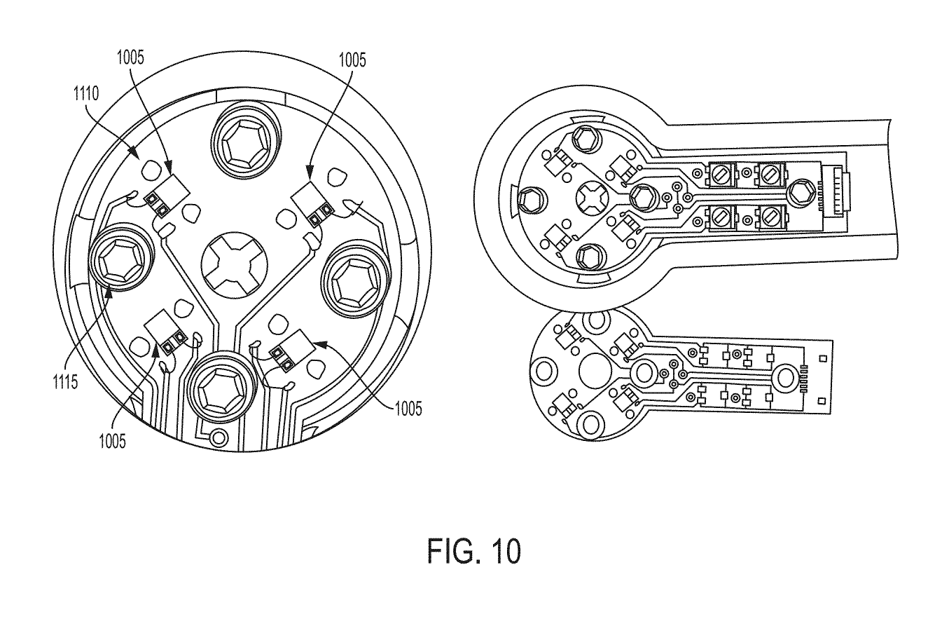

FIG. 10 shows the force sensor, according to examples of the present disclosure. The force sensor includes a resistive strain gauge 1005 that is mounted directly onto the printed circuit board (or force sensor circuit board) and that detects small deflections in the force sensor circuit board caused by finger forces, mounting holes 1010 for the force sensor frame, and mounting screws 1015 that hold the force sensor circuit board onto the force sensor rod. The force sensor frame has pins which pass through these holes in the circuit board, enabling both precision mounting and also a stronger glue bond. The force sensor comprises four pairs of resistive strain gauges (for a total of eight gauges), with each pair being mounted on opposite sides of the board in a half-bridge configuration. Using four sensor pairs to sense forces in three degrees of freedom creates redundancy, which can be used to verify whether the sensor is working properly. Using the circuit board itself as the substrate for the force sensor has the advantage of minimizing the need for long wire leads, since the leads can solder directly onto the circuit board proximal to the sensing elements.

In some examples, other types of force sensors can be used, such as other configurations of strain gauges, optically based sensors, thin film force sensors, load cells, etc. Rather than using force sensor to detect finger force/motion, other types of sensor can be used, such as position sensors like joysticks. Other types of feedback can be given to the user, such as vibration, temperature, pressure, or electrical stimulation. An additional force sensor can be introduced between the wrist support and the chassis, allowing for sensing or wrist forces in addition to finger forces. The finger retention adapters can be implemented in other ways, such as passive or pneumatically powered suction cups, inclusion of rigid parts and spring loading, and removable adhesives. Robotic actuation can be added to the force sensors so that the position of the force sensors can be actively controlled.

In some examples, the device can be interfaced with a computer-driven game, and the force applied to the sensors may be used as input to the game to modify the scenario or any other game parameter. For example, forces/movement of a finger may move a character in the game in three degrees of freedom. In one example, the game can be a maze and the character is moved through the maze in two dimensions by forces/movement of the finger.

FIG. 11 illustrates an example of a hardware configuration for computer device 1100, which can be used to perform one or more of the processes described above. While FIG. 11 illustrates various components contained in computer device 1100, FIG. 11 illustrates one example of a computer device and additional components can be added and existing components can be removed.

Computer device 1100 can be any type of computer devices, such as desktops, laptops, servers, etc., or mobile devices, such as smart telephones, tablet computers, cellular telephones, personal digital assistants, etc. As illustrated in FIG. 11, computer device 1100 can include one or more processors 1102 of varying core configurations and clock frequencies. Computer device 1100 can also include one or more memory devices 1104 that serve as a main memory during the operation of computer device 1100. For example, during operation, a copy of the software that supports the various processing described above can be stored in one or more memory devices 1104. Computer device 1100 can also include one or more peripheral interfaces 1106, such as keyboards, mice, touchpads, computer screens, touchscreens, etc., for enabling human interaction with and manipulation of computer device 1100.

The computer device 1100 can also include one or more network interfaces 1108 for communicating via one or more networks, such as Ethernet adapters, wireless transceivers, or serial network components, for communicating over wired or wireless media using protocols. The computer device 1100 can also include one or more storage device 1110 of varying physical dimensions and storage capacities, such as flash drives, hard drives, random access memory, etc., for storing data, such as images, files, and program instructions for execution by one or more processors 1102.

Additionally, computer device 1100 can include one or more software programs 1112 that enable the functionality described above. One or more software programs 1112 can include instructions that cause one or more processors 1102 to perform the processes described herein. Copies of one or more software programs 1112 can be stored in one or more memory devices 1104 and/or on in one or more storage devices 1110. Likewise, the data used by one or more software programs 1112 can be stored in one or more memory devices 1104 and/or on in one or more storage devices 1110.

In implementations, computer device 1100 can communicate with other devices via network 1116. The other devices can be any types of devices as described above. Network 1116 can be any type of electronic network, such as a local area network, a wide-area network, a virtual private network, the Internet, an intranet, an extranet, a public switched telephone network, an infrared network, a wireless network, and any combination thereof. Network 1116 can support communications using any of a variety of commercially-available protocols, such as TCP/IP, UDP, OSI, FTP, UPnP, NFS, CIFS, AppleTalk, and the like. Network 1116 can be, for example, a local area network, a wide-area network, a virtual private network, the Internet, an intranet, an extranet, a public switched telephone network, an infrared network, a wireless network, and any combination thereof.

Computer device 1100 can include a variety of data stores and other memory and storage media as discussed above. These can reside in a variety of locations, such as on a storage medium local to (and/or resident in) one or more of the computers or remote from any or all of the computers across the network. In some implementations, information can reside in a storage-area network (“SAN”) familiar to those skilled in the art. Similarly, any necessary files for performing the functions attributed to the computers, servers, or other network devices may be stored locally and/or remotely, as appropriate.

In implementations, the components of computer device 1100 as described above need not be enclosed within a single enclosure or even located in close proximity to one another. Those skilled in the art will appreciate that the above-described componentry are examples only, as computer device 1100 can include any type of hardware componentry, including any necessary accompanying firmware or software, for performing the disclosed implementations. Computer device 1100 can also be implemented in part or in whole by electronic circuit components or processors, such as application-specific integrated circuits (ASICs) or field-programmable gate arrays (FPGAs).

If implemented in software, the functions can be stored on or transmitted over a computer-readable medium as one or more instructions or code. Computer-readable media includes both tangible, non-transitory computer storage media and communication media including any medium that facilitates transfer of a computer program from one place to another. A storage media can be any available tangible, non-transitory media that can be accessed by a computer. By way of example, and not limitation, such tangible, non-transitory computer-readable media can comprise RAM, ROM, flash memory, EEPROM, CD-ROM or other optical disk storage, magnetic disk storage or other magnetic storage devices, or any other medium that can be used to carry or store desired program code in the form of instructions or data structures and that can be accessed by a computer. Disk and disc, as used herein, includes CD, laser disc, optical disc, DVD, floppy disk and Blu-ray disc where disks usually reproduce data magnetically, while discs reproduce data optically with lasers. Also, any connection is properly termed a computer-readable medium. For example, if the software is transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. Combinations of the above should also be included within the scope of computer-readable media.

The foregoing description is illustrative, and variations in configuration and implementation can occur to persons skilled in the art. For instance, the various illustrative logics, logical blocks, modules, and circuits described in connection with the embodiments disclosed herein can be implemented or performed with a general purpose processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general-purpose processor can be a microprocessor, but, in the alternative, the processor can be any conventional processor, controller, microcontroller, or state machine. A processor can also be implemented as a combination of computing devices, e.g., a combination of a DSP and a microprocessor, a plurality of microprocessors, one or more microprocessors in conjunction with a DSP core, or any other such configuration.

In one or more exemplary embodiments, the functions described can be implemented in hardware, software, firmware, or any combination thereof. For a software implementation, the techniques described herein can be implemented with modules (e.g., procedures, functions, subprograms, programs, routines, subroutines, modules, software packages, classes, and so on) that perform the functions described herein. A module can be coupled to another module or a hardware circuit by passing and/or receiving information, data, arguments, parameters, or memory contents. Information, arguments, parameters, data, or the like can be passed, forwarded, or transmitted using any suitable means including memory sharing, message passing, token passing, network transmission, and the like. The software codes can be stored in memory units and executed by processors. The memory unit can be implemented within the processor or external to the processor, in which case it can be communicatively coupled to the processor via various means as is known in the art.

While the teachings have been described with reference to examples of the implementations thereof, those skilled in the art will be able to make various modifications to the described implementations without departing from the true spirit and scope. The terms and descriptions used herein are set forth by way of illustration only and are not meant as limitations. In particular, although the processes have been described by examples, the stages of the processes can be performed in a different order than illustrated or simultaneously. Furthermore, to the extent that the terms “including”, “includes”, “having”, “has”, “with”, or variants thereof are used in the detailed description, such terms are intended to be inclusive in a manner similar to the term “comprising.” As used herein, the terms “one or more of” and “at least one of” with respect to a listing of items such as, for example, A and B, means A alone, B alone, or A and B. Further, unless specified otherwise, the term “set” should be interpreted as “one or more.” Also, the term “couple” or “couples” is intended to mean either an indirect or direct connection. Thus, if a first device couples to a second device, that connection can be through a direct connection, or through an indirect connection via other devices, components, and connections.

Those skilled in the art will be able to make various modifications to the described embodiments without departing from the true spirit and scope. The terms and descriptions used herein are set forth by way of illustration only and are not meant as limitations. In particular, although the method has been described by examples, the steps of the method can be performed in a different order than illustrated or simultaneously. Those skilled in the art will recognize that these and other variations are possible within the spirit and scope as defined in the following claims and their equivalents.

The foregoing description of the disclosure, along with its associated embodiments, has been presented for purposes of illustration only. It is not exhaustive and does not limit the disclosure to the precise form disclosed. Those skilled in the art will appreciate from the foregoing description that modifications and variations are possible in light of the above teachings or may be acquired from practicing the disclosure. For example, the steps described need not be performed in the same sequence discussed or with the same degree of separation. Likewise various steps may be omitted, repeated, or combined, as necessary, to achieve the same or similar objectives. Similarly, the systems described need not necessarily include all parts described in the embodiments, and may also include other parts not describe in the embodiments.

Accordingly, the disclosure is not limited to the above-described embodiments, but instead is defined by the appended claims in light of their full scope of equivalents.

What is claimed is:

1. A system for hand rehabilitation comprising:
an enclosure comprising a top surface and a bottom surface opposite the top surface;
a finger retention support arranged on the top surface, sized to accommodate at least a fingertip, and arranged to allow a finger to exert directional movement in three degrees of freedom; and
a first force sensor housed within the enclosure and configured to detect and convert the directional movement exerted by the finger to one or more electrical control signals.

2. The system of claim 1, further comprising:
a wrist support attached to the top surface and configured to support and rigidly hold a wrist in position with respect to the top surface; and
a processor in electrical communication with the first force sensor and configured to receive the one or more electrical control signals from the first force sensor and provide feedback to a user.

3. The system of claim 1, further comprising:
a printed circuit board (PCB) housed within the enclosure, wherein the first force sensor is directly mounted on the PCB.

4. The system of claim 3, further comprising a second force sensor, a third force sensor, and a fourth force sensor mounted directly on the PCB.

5. The system of claim 3, wherein the first force sensor, the second force sensor, the third force sensor, and the fourth force sensor are mounted on a top surface of the PCB.

6. The system of claim 3, further comprising a fifth force sensor, a sixth force sensor, a seventh force sensor, and an eighth force sensor mounted directly on a bottom surface of the PCB.

7. The system of claim 1, wherein the first force sensor is a strain gauge.

8. The system of claim 2, further comprising a display configured to display the feedback.

9. The system of claim 2, wherein the processor is configured to perform operations based on movements of the finger as measured by the first force sensor.

10. The system of claim 1, wherein the feedback comprises an interactive video or video game.

11. The system of claim 1, wherein the finger retention support comprises an elastic silicon cup bonded onto the top surface, a passive retention support, a pneumatic powered suction cup, a rigid support with one or more elastic or spring loaded parts which exert pressure on the finger, a rigid support that can be mechanically adjusted in tightness around the finger such as a by a collet mechanism or a screw/band clamp mechanism, or a support with removable adhesive.

12. The system of claim 1, wherein the finger retention support comprises one finger retention support for each finger.

13. The system of claim 1, wherein the first force sensor is configured to detect bi-directional forces exerted by the finger on the finger retention support in multiple degrees of freedom.

14. A system for hand rehabilitation comprising:
an enclosure comprising a top surface and a bottom surface opposite the top surface;
an input device arranged on the top surface and arranged to allow a finger to exert directional movement in three degrees of freedom; and
a first force sensor housed within the enclosure and configured to detect and convert the directional movement exerted by the finger to one or more electrical control signals.

15. The system of claim 14, further comprising:
a wrist support attached to the top surface and configured to support and rigidly hold a wrist in position with respect to the top surface; and
a processor in electrical communication with the first force sensor and configured to receive the one or more electrical control signals from the first force sensor and provide feedback to a user.

16. The system of claim 14, further comprising:
a printed circuit board (PCB) housed within the enclosure, wherein the first force sensor is directly mounted on a top surface of the PCB.

17. The system of claim 16, further comprising a second force sensor, a third force sensor, and a fourth force sensor mounted directly on the top surface of the PCB.

18. The system of claim 16, further comprising a fifth force sensor, a sixth force sensor, a seventh force sensor, and an eighth force sensor mounted directly on a bottom surface of the PCB.

19. The system of claim 14, wherein the first force sensor is a strain gauge.
